(19) United States
(12) Patent Application Publication
(10) Pub. No.: US 2024/0350084 A1
(43) Pub. Date: Oct. 24, 2024

(54) ADVICE GENERATION APPARATUS, ADVICE GENERATION METHOD, AND NON-TRANSITORY COMPUTER-READABLE STORAGE MEDIUM

(71) Applicant: NEC Corporation, Minato-ku, Tokyo (JP)

(72) Inventors: Nirmal Singh RAJPUT, Tokyo (JP); Nobuyuki YASUKAWA, Tokyo (JP); Mion ABE, Minato-ku (JP); Hiromi SHIMZU, Tokyo (JP); Hiroki OGAWA, Tokyo (JP); Masahiro KUNIEDA, Tokyo (JP)

(73) Assignee: NEC Corporation, Miinato-ku, Tokyo (JP)

(21) Appl. No.: 18/566,251

(22) PCT Filed: Jun. 4, 2021
(86) PCT No.: PCT/JP2021/021395

§ 371 Date: Dec. 1, 2023

(51) Int. Cl. A61B 5/00; A61B 5/145; G16H 20/60

(52) U.S. Cl. CPC A61B 5/486; A61B 5/14532; A61B 5/4866; G16H 20/60

(57) ABSTRACT

An advice generation apparatus includes: an information acquisition unit that acquires a first blood glucose level being a blood glucose level of a subject at a first timing, meal information relating to a meal to be ingested by the subject later than the first timing, and a reference range of the blood glucose level of the subject; a rule acquisition unit that acquires a blood glucose level estimation rule which is set for each subject and estimates, using the first blood glucose level and the meal information, a second blood glucose level being a blood glucose level at a second timing after ingesting the meal; a blood glucose level estimation unit that estimates the second blood glucose level using the first blood glucose level, the meal information, and the blood glucose level estimation rule; and an advice generation unit that generates advice information including advice relating to the meal.

### TECHNICAL FIELD

The present invention relates to an advice generation apparatus, an advice generation method, and a program.

### BACKGROUND ART

A blood glucose level, for example, a glucose level in blood is important for managing person's health. In order to recognize a transition of daily blood glucose levels in a person, generally, it is necessary to measure a blood glucose level a plurality of times during a day. In regard to this, Patent Document 1 describes that a blood glucose level measured before breakfast is used for predicting a blood glucose level and outputting a predicted blood glucose level curve, each time a meal is ingested and meal information is input, and each time calories burned by a user are measured.

### RELATED DOCUMENT

### Patent Document

Patent Document 1: Japanese Patent Application Publication No. 2012-181804

### SUMMARY OF INVENTION

### Technical Problem

A preferred range of blood glucose levels may vary from person to person due to presence or absence of a pre-existing medical condition or the like. Further, a blood glucose level varies depending on a content of a meal or an amount of post-meal exercise. Thus, it is difficult for a person to keep his or her own blood glucose level within a preferred range. One example of an object of the present invention is to make it easier for a person to keep his or her own blood glucose level within a preferred range.

### Solution to Problem

According to one aspect of the present invention, provided is an advice generation apparatus including:
an information acquisition unit that acquires a first blood glucose level being a blood glucose level of a subject at a first timing, meal information being information relating to an item of a meal to be ingested by the subject later than the first timing, and a reference range of a blood glucose level of the subject;
a rule acquisition unit that acquires a blood glucose level estimation rule being set for each of the subjects and estimating, by using the first blood glucose level and the meal information, a second blood glucose level being a blood glucose level at a second timing after the subject acquires the meal;
a blood glucose level estimation unit that estimates the second blood glucose level by using the first blood glucose level, the meal information, and the blood glucose level estimation rule; and
an advice generation unit that generates, by using the reference range and the second blood glucose level, advice information including at least one of advice relating to the meal and advice relating to behavior of the subject after ingesting the meal.

According to the present invention, provided is an advice generation method including
performing, by a computer:
information acquisition processing of acquiring a first blood glucose level being a blood glucose level of a subject at a first timing, meal information being information relating to a meal to be ingested by the subject later than the first timing, and a reference range of a blood glucose level of the subject;
rule acquisition processing of acquiring a blood glucose level estimation rule being set for each of the subjects and estimating, by using the first blood glucose level and the meal information, a second blood glucose level being a blood glucose level at a second timing after the subject acquires the meal;
blood glucose level estimation processing of estimating the second blood glucose level by using the first blood glucose level, the meal information, and the blood glucose level estimation rule; and
advice generation processing of generating, by using the reference range and the second blood glucose level, advice information including at least one of advice relating to the meal and advice relating to behavior of the subject after ingesting the meal.

According to the present invention, provided is a program causing a computer to have:
an information acquisition function of acquiring a first blood glucose level being a blood glucose level of a subject at a first timing, meal information being information relating to a meal to be ingested by the subject later than the first timing, and a reference range of a blood glucose level of the subject;
a rule acquisition function of acquiring a blood glucose level estimation rule being set for each of the subjects and estimating, by using the first blood glucose level and the meal information, a second blood glucose level being a blood glucose level at a second timing after the subject acquires the meal;
a blood glucose level estimation function of estimating the second blood glucose level by using the first blood glucose level, the meal information, and the blood glucose level estimation rule; and
an advice generation function of generating, by using the reference range and the second blood glucose level, advice information including at least one of advice relating to the meal and advice relating to behavior of the subject after ingesting the meal.

### Advantageous Effects of Invention

According to one aspect of the present invention, a person is able to easily keep his or her own blood glucose level within a preferred range.

### BRIEF DESCRIPTION OF THE DRAWINGS

The above-described objective and other objectives, features and advantages are more apparent from the following preferred example embodiment and the accompanying drawings.

FIG. 1 It is a diagram for describing a usage environment of an advice generation apparatus according to an example embodiment.

FIG. 2 It is a diagram illustrating one example of a function configuration of the advice generation apparatus.

FIG. 3 It is a diagram illustrating one example of information acquired by an information acquisition unit.

FIG. 4 It is a diagram illustrating a first example of information stored in a storage unit.

FIG. 5 It is a diagram illustrating a second example of information stored in the storage unit.

FIG. 6 It is a diagram illustrating a hardware configuration example of the advice generation apparatus.

FIG. 7 It is a flowchart illustrating one example of processing performed by the advice generation apparatus.

### EXAMPLE EMBODIMENT

Hereinafter, an example embodiment of the present invention will be described by using the drawings. Note that, a similar component is assigned with a similar reference sign throughout all the drawings, and description therefor will be not included as appropriate.

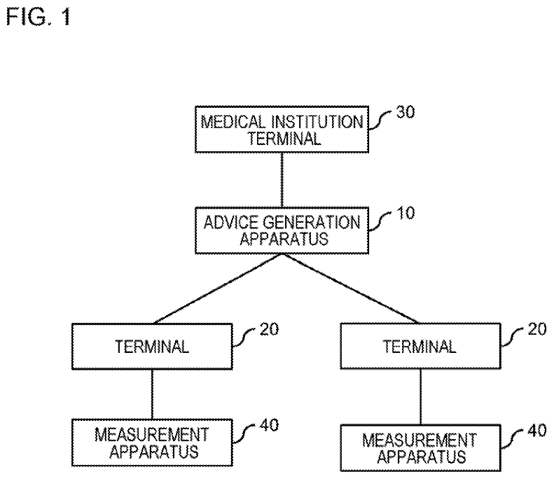

FIG. 1 is a diagram for describing a usage environment of an advice generation apparatus 10. The advice generation apparatus 10 is an apparatus that provides a subject with advice for keeping a blood glucose level of the subject within a predetermined range, and is used together with a terminal 20. The terminal 20 is connected to the advice generation apparatus 10 via, for example, an internet communication network.

The terminal 20 is operated by a subject. Specifically, a subject measures, by using a measurement apparatus 40, a blood glucose level (hereinafter, written as a first blood glucose level) at a predetermined timing (hereinafter, written as a first timing) in a day. There is, for example, one first timing per day, but there may be a plurality of first timings. The measurement apparatus 40 is, for example, but not limited to, a wearable device. Then, the subject transmits the first blood glucose level to the advice generation apparatus 10 via the terminal 20. The first timing is, for example, but not limited to, at least one of before breakfast and before bedtime. Further, the subject transmits, before taking a meal, information relating to the meal (hereinafter, written as meal information) to the advice generation apparatus 10 via the terminal 20.

Then, the advice generation apparatus 10 estimates a blood glucose level (hereinafter, written as a second blood glucose level) at a predetermined timing (hereinafter, written as a second timing) after acquiring the meal. There may be a plurality of second timings. Then, the advice generation apparatus 10 generates advice information by using the second blood glucose level and a reference range of a blood glucose level of the subject. The advice information includes at least one of advice relating to the meal and advice relating to behavior of the subject after ingesting the meal.

Then, the advice generation apparatus 10 transmits the generated advice information to the terminal 20. At this time, the advice generation apparatus 10 may transmit the second blood glucose level to the advice generation apparatus 10 together with the advice information or instead of the advice information. In a latter case, the advice generation apparatus 10 may not generate the advice information.

Note that, the advice generation apparatus 10 has a plurality of subjects. In addition, each of the plurality of subjects has the terminal 20. Thus, a plurality of terminals 20 are connected to one advice generation apparatus 10.

Further, the advice generation apparatus 10 may be further connected to a medical institution terminal 30. In this case, the advice generation apparatus 10 transmits the first blood glucose level and the second blood glucose level to the medical institution terminal 30. The advice generation apparatus 10 may be further connected to a terminal managed by a meal provider, or may be connected to a terminal managing a food item inventory of the subject.

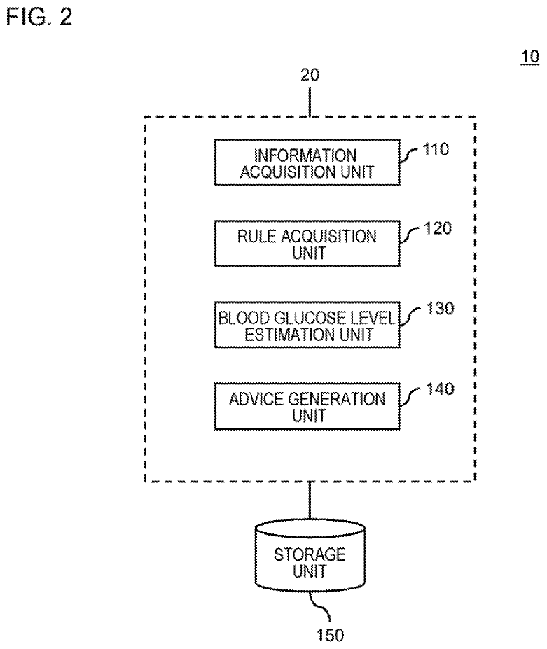

FIG. 2 is a diagram illustrating one example of a function configuration of the advice generation apparatus 10. The advice generation apparatus 10 includes an information acquisition unit 110, a rule acquisition unit 120, a blood glucose level estimation unit 130, an advice generation unit 140, and a storage unit 150. The storage unit 150 stores various kinds of information necessary for processing performed by the advice generation apparatus 10.

The information acquisition unit 110 acquires, from the terminal 20 and the storage unit 150, information necessary for processing performed by the advice generation apparatus 10.

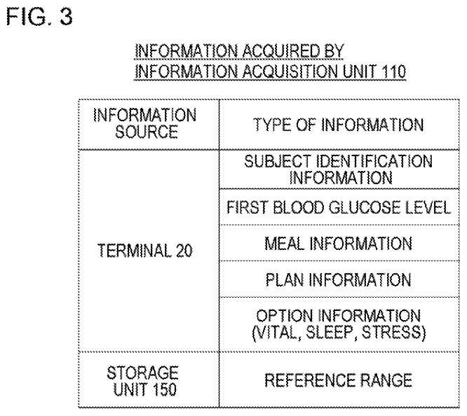

FIG. 3 is a diagram illustrating one example of information acquired by the information acquisition unit 110. The information acquisition unit 110 acquires, from the terminal 20, the first blood glucose level and the meal information together with information identifying a subject (hereinafter, written as subject identification information).

The first blood glucose level is an actual measurement value of a blood glucose level at the first timing, as described above. Here, only one first timing may be set in a day, or two or more first timings may be set. In a latter case, a plurality of first timings are, for example, at least two of before breakfast, before lunch, and before dinner. The terminal 20 may acquire the first blood glucose level from the measurement apparatus 40 via wireless communication, or may acquire the first blood glucose level through an input from a subject.

The meal information includes, for example, at least one of an item included in the meal and information capable of determining calories to be ingested in the meal (for example, a value of calories per se, or a quantity of nutrients (for example, a carbohydrate, a fat, and a protein) contained in the meal). For example, the meal information may be an image of the actual meal, or may include information identifying an item (hereinafter, written as item identification information). The item identification information may be an item name, may be an image containing a character or a sentence indicating a meal content, or may be code information indicating a meal content. Here, the item name is input by, for example, a subject. Further, the sentence indicating a meal content may be prepared by, for example, a meal provider (for example, a proprietor of a restaurant). Further, the code information may be a code (for example, a barcode or a two-dimensional code) assigned to food packaging, or may be a code assigned to a menu.

Further, the information acquisition unit 110 may further acquire plan information and option information.

The plan information indicates an activity schedule of a subject on the day, and is input to the terminal 20 by the subject.

For example, the plan information includes a content of a meal to be acquired later than a meal indicated by the meal information (for example, a content of dinner when the meal information indicates lunch), and an activity schedule on the day (for example, whether a party is held at night on the day). These pieces of information are used to compute a quantity of calories that may be ingested in a meal indicated by the meal information acquired this time. For example, when the meal information indicates lunch, the blood glucose level estimation unit 130 to be described later computes, by using a content of dinner, calories that may be acquired in lunch. The calories computed herein are used in estimating the second blood glucose level. Further, when a party is held at night on the day, the blood glucose level estimation unit 130 lowers calories that may be acquired in lunch.

Further, the plan information may include an inventory of food saved in a pantry or a refrigerator, and food to be delivered by a deliverer on the day. These pieces of information are used in estimating a content of a meal (for example, dinner) to be acquired later than a meal indicated by the meal information. The content of a meal estimated herein is used for computing a quantity of calories that may be ingested in a meal indicated by the meal information acquired this time, as described above.

The option information is information that is not essential but is preferable if available. One example of the option information is at least one of vital information, sleep information, and stress information. The vital information includes, for example, at least one of a heart rate, a blood pressure, and a respiratory rate. The sleep information includes at least information indicating sleep time (for example, a bedtime and a wake-up time), and further includes information indicating quality of sleep (for example, a chart indicating sleep stages). The stress information is a value indicating strength of stress. As one example, “1” indicates being relaxed, “5” indicates being in a normal state, and “10” indicates being stressed. These pieces of option information may be input to the terminal 20 by a subject, or data measured by the measurement apparatus 40 may be wirelessly transmitted from the measurement apparatus to the terminal 20. Further, the stress information may be generated from the vital information, or may be a result of judgement by an expert such as a doctor.

The information acquisition unit 110 further acquires, from the storage unit 150, a range (hereinafter, a reference range) that a blood glucose level of the subject needs to take. The reference range is set for each subject. The reason is that an adequate range of a blood glucose level varies depending on presence or absence of a pre-existing medical condition (for example, diabetes or the like) or the like. Thus, the storage unit 150 stores the reference range in association with the subject identification information of a subject relevant to the reference range. Then, the information acquisition unit 110 reads out, from the storage unit 150, the reference range associated with the subject identification information acquired from the terminal 20.

Return to FIG. 2. The rule acquisition unit 120 acquires, from the storage unit 150, a rule for estimating the second blood glucose level described above (hereinafter, written as a blood glucose level estimation rule). The blood glucose level estimation rule is a rule for estimating the second blood glucose level by using the first blood glucose level and the meal information. The rule may be, for example, a model generated by using machine learning such as a neural network, or may be defined in a rule-based way such as a computational expression. The second blood glucose level is a blood glucose level at the second timing, as described above.

Here, when the meal information relates to an item of breakfast, the second timing is a period between breakfast and lunch. Further, when the meal information relates to an item of lunch, the second timing is a period between lunch and dinner. Further, when the meal information relates to an item of dinner, the second timing may be a period between dinner and bedtime, or may be during bedtime. In addition, the second blood glucose level is a representative value for a blood glucose level in the period. Further, a plurality of second timings may be set in at least one of the periods described above. For example, when it is defined that “the second timing is after a lapse of reference time after a meal is finished (or started)”, a plurality of the reference times may be set in a period before a subject takes a next meal.

The blood glucose level estimation rule is set for each subject. Thus, the storage unit 150 stores the blood glucose level estimation rule in association with the subject identification information of a subject relevant to the blood glucose level estimation rule. Then, the information acquisition unit 110 reads out, from the storage unit 150, the blood glucose level estimation rule associated with the subject identification information acquired from the terminal 20.

The blood glucose level estimation unit 130 estimates the second blood glucose level by using the first blood glucose level and the meal information acquired by the information acquisition unit 110 and the blood glucose level estimation rule acquired by the rule acquisition unit 120.

Note that, the blood glucose level estimation rule may use at least one of following items as an input parameter, in addition to the first blood glucose level and the meal information.
A history of first blood glucose levels (past first blood glucose levels)
At least one of an actual measurement value of a blood glucose level before lunch and an actual measurement value of a blood glucose level before dinner (when the first blood glucose level is a blood glucose level before breakfast)
At least one of an actual measurement value of a blood glucose level after breakfast, an actual measurement value of a blood glucose level after lunch, and an actual measurement value of a blood glucose level after dinner (when the first blood glucose level is a blood glucose level before breakfast).
A content of post-meal exercise and time thereof (including a case of doing no exercise)

Here, one example of the blood glucose level estimation rule will be described. In the example, the first blood glucose level is a blood glucose level before breakfast (that is, a fasting blood glucose level). Further, the meal information includes information capable of determining calories to be ingested in the meal. In addition, the blood glucose level estimation rule is rule-based, as indicated below.

$$The second blood glucose level = the first blood glucose level + ( calories to be ingested in the meal ) \times α$$

where α is a coefficient determined by using the first blood glucose level and the meal information, and is set for each ingestion timing of the meal (for example, breakfast, lunch, and dinner). In computing α, following information may be further used.
Ingestion order of items included in the meal
Quantity of calories of a meal ingested earlier than the meal being treated this time in the same day

A rule for computing a is set for each ingestion timing of the meal by using a past actual value of the user. A rule for computing a may be rule-based, or may be a model using machine learning.

The advice generation unit 140 generates the advice information described above, by using the reference range acquired by the information acquisition unit 110 and the second blood glucose level estimated by the blood glucose level estimation unit 130. As one example, the advice generation unit 140 includes, in the advice information, advice relating to a meal when a blood glucose level deviates from the reference range.

For example, when the second blood glucose level goes above the reference range, that is, when the second blood glucose level exceeds an upper limit value of the reference range, the advice generation unit 140 generates, as the advice information, information recommending at least one of reducing an ingestion amount of a specific item (a first item) and not ingesting a specific item (a second item), as at least a part of the advice information.

Further, when the second blood glucose level falls below the reference range, that is, when the second blood glucose level exceeds a lower limit value of the reference range, the advice generation unit 140 generates, as the advice information, information indicating increasing a meal amount, for example, information recommending at least one of increasing an ingestion amount of a specific item and adding a specific amount of a new item, as at least a part of the advice information.

Further, the advice generation unit 140 may generate, as the advice information, information indicating that a meal itself must not be taken.

Note that, the advice generation unit 140 may generate the advice information according to a rule-based way, or may generate the advice information by using a model generated by machine learning. In a former case, the advice generation unit 140 generates the advice information, for example, in a way as follows. A quantity of calories that needs to be increased/decreased in a meal of this time is computed by using a difference between the reference range and the second blood glucose level. Then, the advice generation unit 140 determines an item equivalent to the calories and an amount thereof, and generates the advice information by using the determined amount.

Further, when a plurality of items are included in a meal, the advice generation unit 140 may include, in the advice information, ingestion order of the plurality of items. In this case, the advice generation unit 140 may include, in the advice information, an ingestion interval between two items adjacent to each other in ingestion order. The ingestion interval indicates, for example, that “a next item is eaten after a lapse of predetermined time (for example, five minutes) after a certain item is eaten”. The advice generation unit 140 generates the advice information according to a rule-based way. Note that, the advice information may not be based on the second blood glucose level.

Further, the advice generation unit 140 may include, in the advice information, at least one of the second blood glucose level and information indicating whether the second blood glucose level falls within the reference range. For example, the advice generation unit 140 may include, in the advice information, a graph indicating the second blood glucose level and the reference range.

Note that, a subject may desire to ingest a meal as is. When assuming to comply with the desire, the second blood glucose level estimated by the blood glucose level estimation unit 130 may deviate from the reference range. In this case, the advice generation unit 140 may include, in the advice information, advice relating to behavior of the subject after ingesting the meal. The advice is intended to shorten time during which a blood glucose level deviates from the reference range. Specifically, the advice information may include an item of exercise (for example, walking, running, swimming, or the like) that needs to be performed after the meal and exercise time thereof, or may include an item of relaxation (for example, meditation or sleep) that needs to be performed after the meal and duration time thereof. Further, the advice generation unit 140 may generate the advice information relating to the meal in such a way as to shorten time during which a blood glucose level deviates from the reference range.

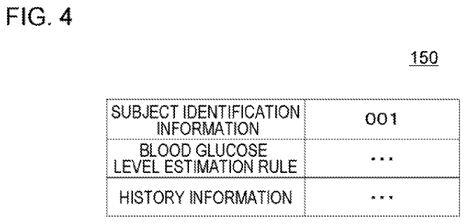

FIG. 4 is a diagram illustrating a first example of information stored in the storage unit 150. The storage unit 150 stores, for each of a plurality of subjects, the subject identification information, the blood glucose level estimation rule or information for generating the rule, and history information, as illustrated in the present figure. The history information includes an item included in a meal, a date and time in which the meal is ingested, an actual measurement value of a blood glucose level, and a date and time of measuring the blood glucose level. Note that, the history information is used in updating the blood glucose level estimation rule.

More specifically, the blood glucose level estimation unit 130 regularly updates the blood glucose level estimation rule. For example, a subject repeats, for example, for one week, measuring a blood glucose level a plurality of times during a day for every predetermined period (for example, every one month, every three months, or every six months). A measurement timing of a blood glucose level is, for example, before breakfast, after breakfast, before lunch, after lunch, before dinner, after dinner, and before bedtime. These pieces of data are transmitted to the advice generation apparatus 10 via the terminal 20, and are stored in the storage unit 150 as a part of the history information. At this time, the terminal 20 also acquires the meal information, the plan information, and the option information. Furthermore, the terminal 20 may also acquire information relating to exercise performed on the day. These pieces of information are also stored in the storage unit 150 as a part of the history information. Then, the blood glucose level estimation unit 130 updates the blood glucose level estimation rule by using these pieces of the history information.

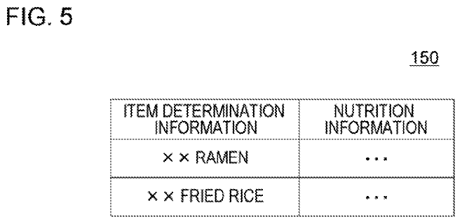

FIG. 5 is a diagram illustrating a second example of information stored in the storage unit 150. The storage unit 150 stores, for each item, the item identification information of the item and information determining a quantity of calories included in the item, as illustrated in the present figure. One example of the information determining a quantity of calories is nutrients contained in the item and a quantity thereof. The nutrients stored in the storage unit 150 include at least a carbohydrate, a fat, and a protein. However, the storage unit 150 may store the quantity of calories instead of the nutrients and the quantity thereof, or in addition thereto.

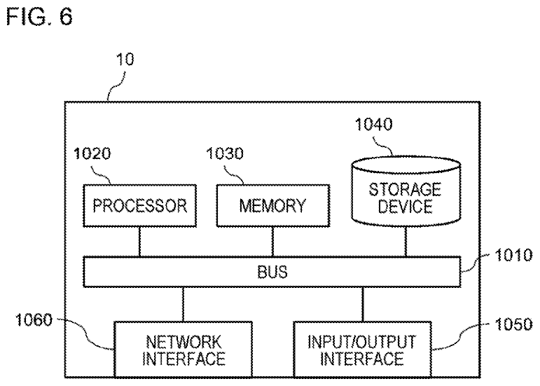

FIG. 6 is a diagram illustrating a hardware configuration example of the advice generation apparatus 10. The advice generation apparatus 10 includes a bus 1010, a processor 1020, a memory 1030, a storage device 1040, an input/output interface 1050, and a network interface 1060.

The bus 1010 is a data transmission path through which the processor 1020, the memory 1030, the storage device 1040, the input/output interface 1050, and the network interface 1060 transmit and receive data to and from one another. However, a method of connecting the processor 1020 and the like with one another is not limited to bus connection.

The processor 1020 is a processor achieved by a central processing unit (CPU), a graphics processing unit (GPU), or the like.

The memory 1030 is a main storage apparatus achieved by a random access memory (RAM) or the like.

The storage device 1040 is an auxiliary storage apparatus achieved by a hard disk drive (HDD), a solid state drive (SSD), a memory card, a read only memory (ROM), or the like. The storage device 1040 stores program modules for achieving functions (for example, the information acquisition unit 110, the rule acquisition unit 120, the blood glucose level estimation unit 130, and the advice generation unit 140) of the advice generation apparatus 10. Each of the program modules is read in to the memory 1030 and executed by the processor 1020, and thereby each function relevant to the program module is achieved. Further, the storage device 1040 also functions as the storage unit 150.

The input/output interface 1050 is an interface for connecting the advice generation apparatus 10 to various kinds of input/output equipment.

The network interface 1060 is an interface for connecting the advice generation apparatus 10 to a network. The network is, for example, a local area network (LAN) or a wide area network (WAN). A method by which the network interface 1060 connects to a network may be wireless communication, or may be wired communication. The advice generation apparatus 10 communicates with the terminal 20 or other terminals (for example, the medical institution terminal 30) via the network interface 1060.

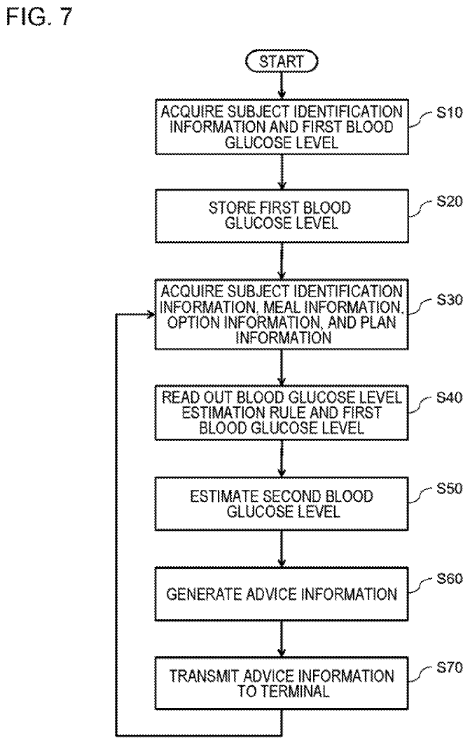

FIG. 7 is a flowchart illustrating one example of processing performed by the advice generation apparatus 10. The advice generation apparatus 10 repeats processing illustrated in the present figure, for example, on a daily basis.

First, a subject measures a first blood glucose level at a specific timing (for example, after waking up, before breakfast). In this case, the first blood glucose level is a fasting blood glucose level. Then, the terminal 20 acquires the first blood glucose level, and transmits the first blood glucose level to the advice generation apparatus 10 together with subject identification information. Note that, the subject identification information may be stored in advance in the terminal 20, or may be input to the terminal 20 by the subject at a time of transmission.

Upon acquiring the first blood glucose level and the subject identification information transmitted by the terminal 20 (Step S10), the information acquisition unit 110 of the advice generation apparatus 10 causes the storage unit 150 to store, as a part of history information, the first blood glucose level in association with the subject identification information (Step S20). Then, the rule acquisition unit 120, the blood glucose level estimation unit 130, and the advice generation unit 140 perform processing indicated in Steps S20 to S70 each time meal information or the like is acquired from the terminal 20.

Specifically, the subject inputs at least the meal information to the terminal 20 before ingesting a meal. At this time, the subject may input, to the terminal 20, at least one of option information and plan information. Then, the terminal 20 transmits the input information to the advice generation apparatus 10 in association with the subject identification information.

The advice generation apparatus 10 and the information acquisition unit 110 acquire the information transmitted by the terminal 20 (Step S30). Then, the information acquisition unit 110 reads out, from the storage unit 150, the first blood glucose level relevant to the subject identification information acquired in Step S30. Further, the rule acquisition unit 120 reads out, from the storage unit 150, a blood glucose level estimation rule relevant to the subject identification information acquired in Step S30 (Step S40).

Then, the blood glucose level estimation unit 130 inputs, to the blood glucose level estimation rule acquired in Step S40, the first blood glucose level acquired in Step S40 and the information acquired in Step S30, thereby estimating a second blood glucose level (Step S50). Then, the advice generation unit 140 generates advice information by using the second blood glucose level generated in Step S50 (Step S60), and transmits the generated advice information to the terminal 20 (Step S70).

Thereafter, the blood glucose level estimation unit 130 causes the storage unit 150 to store the second blood glucose level in association with the subject identification information acquired in Step S30. Further, the advice generation unit 140 may cause the storage unit 150 to store the advice information in association with the subject identification information acquired in Step S30.

Further, the advice generation apparatus 10 may transmit the first blood glucose level and the second blood glucose level to the medical institution terminal 30. In this case, medical personnel can recognize the first blood glucose level and the second blood glucose level.

As described above, according to the present example embodiment, the advice generation apparatus 10 generates advice information before a subject ingests a meal. The advice information includes at least one of advice relating to the meal and advice relating to behavior of the subject after ingesting the meal. Accordingly, a person can easily keep his or her own blood glucose level within a preferred range.

While the example embodiment of the present invention has been described with reference to the drawings, the example embodiment is illustrative of the present invention, and various configurations other than the above can be employed.

Further, while a plurality of processes (pieces of processing) are described in order in a plurality of flowcharts used in the above description, execution order of processes executed in each example embodiment is not limited to the described order. The order of the illustrated processes can be changed in each example embodiment, as long as the change does not detract from contents. Further, the above example embodiments can be combined, as long as contents do not contradict each other.

The whole or part of the above-described example embodiment can be described as, but not limited to, the following supplementary notes.
1. An advice generation apparatus including:
   an information acquisition unit that acquires a first blood glucose level being a blood glucose level of a subject at a first timing, meal information being information relating to a meal to be ingested by the subject later than the first timing, and a reference range of a blood glucose level of the subject;
   a rule acquisition unit that acquires a blood glucose level estimation rule being set for each of the subjects and estimating, by using the first blood glucose level and the meal information, a second blood glucose level being a blood glucose level at a second timing after the subject acquires the meal;
   a blood glucose level estimation unit that estimates the second blood glucose level by using the first blood glucose level, the meal information, and the blood glucose level estimation rule; and
   an advice generation unit that generates, by using the reference range and the second blood glucose level, advice information including at least one of advice relating to the meal and advice relating to behavior of the subject after ingesting the meal.
2. The advice generation apparatus according to supplementary note 1, wherein
   a plurality of the second timings are set, and
   the blood glucose level estimation unit estimates the second blood glucose level at each of the plurality of second timings.
3. The advice generation apparatus according to supplementary note 1 or 2, wherein
   a plurality of the first timings are set, and
   the information acquisition unit acquires the first blood glucose level at each of the plurality of first timings.
4. The advice generation apparatus according to any one of supplementary notes 1 to 3, wherein
   the advice generation unit generates the advice information including advice relating to the meal when the second blood glucose level deviates from the reference range.
5. The advice generation apparatus according to supplementary note 4, wherein
   the advice information includes information indicating not ingesting a first item included in the meal.
6. The advice generation apparatus according to supplementary note 4 or 5, wherein
   the advice information includes an ingestion amount of a second item included in the meal.
7. The advice generation apparatus according to any one of supplementary notes 1 to 6, wherein
   the meal includes a plurality of items, and
   the advice information includes ingestion order of the plurality of items.
8. The advice generation apparatus according to supplementary note 7, wherein
   the advice information further includes an ingestion interval between two of the items adjacent to each other in ingestion order.
9. The advice generation apparatus according to any one of supplementary notes 1 to 8, wherein
   the first blood glucose level is a blood glucose level before breakfast,
   the meal information includes an item included in the meal and also includes information capable of determining calories to be ingested in the meal, and
   the blood glucose level estimation rule is indicated by a following equation.

$$The second blood glucose level = the first blood glucose level + ( calories to be ingested in the meal ) \times α$$

where α is a coefficient computed by using the first blood glucose level and the meal information.
10. An advice generation method including
   performing, by a computer:
   information acquisition processing of acquiring a first blood glucose level being a blood glucose level of a subject at a first timing, meal information being information relating to a meal to be ingested by the subject later than the first timing, and a reference range of a blood glucose level of the subject;
   rule acquisition processing of acquiring a blood glucose level estimation rule being set for each of the subjects and estimating, by using the first blood glucose level and the meal information, a second blood glucose level being a blood glucose level at a second timing after the subject acquires the meal;
   blood glucose level estimation processing of estimating the second blood glucose level by using the first blood glucose level, the meal information, and the blood glucose level estimation rule; and
   advice generation processing of generating, by using the reference range and the second blood glucose level, advice information including at least one of advice relating to the meal and advice relating to behavior of the subject after ingesting the meal.
11. The advice generation method according to supplementary note 10, wherein
   a plurality of the second timings are set, and
   the computer estimates, in the blood glucose level estimation processing, the second blood glucose level at each of the plurality of second timings.
12. The advice generation method according to supplementary note 10 or 11, wherein
   a plurality of the first timings are set, and
   the computer acquires, in the information acquisition processing, the first blood glucose level at each of the plurality of first timings.
13. The advice generation method according to any one of supplementary notes 10 to 12, wherein
   the computer generates, in the advice generation processing, the advice information including advice relating to the meal when the second blood glucose level deviates from the reference range.
14. The advice generation method according to supplementary note 13, wherein
   the advice information includes information indicating not ingesting a first item included in the meal.
15. The advice generation method according to supplementary note 13 or 14, wherein
   the advice information includes an ingestion amount of a second item included in the meal.
16. The advice generation method according to any one of supplementary notes 10 to 15, wherein
   the meal includes a plurality of items, and
   the advice information includes ingestion order of the plurality of items.
17. The advice generation method according to supplementary note 16, wherein
   the advice information further includes an ingestion interval between two of the items adjacent to each other in ingestion order.
18. The advice generation method according to any one of supplementary notes 10 to 17, wherein
   the first blood glucose level is a blood glucose level before breakfast,
   the meal information includes an item included in the meal and also includes information capable of determining calories to be ingested in the meal, and
   the blood glucose level estimation rule is indicated by a following equation.

$$The second blood glucose level = the first blood glucose level + ( calories to be ingested in the meal ) \times α$$

where α is a coefficient computed by using the first blood glucose level and the meal information.
19. A program causing a computer to have:
   an information acquisition function of acquiring a first blood glucose level being a blood glucose level of a subject at a first timing, meal information being information relating to a meal to be ingested by the subject later than the first timing, and a reference range of a blood glucose level of the subject;
   a rule acquisition function of acquiring a blood glucose level estimation rule being set for each of the subjects and estimating, by using the first blood glucose level and the meal information, a second blood glucose level being a blood glucose level at a second timing after the subject acquires the meal;
   a blood glucose level estimation function of estimating the second blood glucose level by using the first blood glucose level, the meal information, and the blood glucose level estimation rule; and
   an advice generation function of generating, by using the reference range and the second blood glucose level, advice information including at least one of advice relating to the meal and advice relating to behavior of the subject after ingesting the meal.
20. The program according to supplementary note 19, wherein
   a plurality of the second timings are set, and
   the blood glucose level estimation function estimates the second blood glucose level at each of the plurality of second timings.
21. The program according to supplementary note 19 or 20, wherein
   a plurality of the first timings are set, and
   the information acquisition function acquires the first blood glucose level at each of the plurality of first timings.
22. The program according to any one of supplementary notes 19 to 21, wherein
   the advice generation function generates the advice information including advice relating to the meal when the second blood glucose level deviates from the reference range.
23. The program according to supplementary note 22, wherein
   the advice information includes information indicating not ingesting a first item included in the meal.
24. The program according to supplementary note 22 or 23, wherein
   the advice information includes an ingestion amount of a second item included in the meal.
25. The program according to any one of supplementary notes 19 to 24, wherein
   the meal includes a plurality of items, and
   the advice information includes ingestion order of the plurality of items.
26. The program according to supplementary note 25, wherein
   the advice information further includes an ingestion interval between two of the items adjacent to each other in ingestion order.
27. The program according to any one of supplementary notes 19 to 26, wherein
   the first blood glucose level is a blood glucose level before breakfast,
   the meal information includes an item included in the meal and also includes information capable of determining calories to be ingested in the meal, and
   the blood glucose level estimation rule is indicated by a following equation.

$$The second blood glucose level = the first blood glucose level + ( calories to be ingested in the meal ) \times α$$

where α is a coefficient computed by using the first blood glucose level and the meal information.

### REFERENCE SIGNS LIST

10 Advice generation apparatus
20 Terminal
30 Medical institution terminal
40 Measurement apparatus
110 Information acquisition unit
120 Rule acquisition unit
130 Blood glucose level estimation unit
140 Advice generation unit
150 Storage unit

What is claimed is:

1. An advice generation apparatus comprising:
at least one memory configured to store instructions; and
at least one processor configured to execute the instructions to:
acquire a first blood glucose level being a blood glucose level of a subject at a first timing, meal information being information relating to a meal to be ingested by the subject later than the first timing, and a reference range of a blood glucose level of the subject;
acquire a blood glucose level estimation rule being set for each of the subjects and estimating, by using the first blood glucose level and the meal information, a second blood glucose level being a blood glucose level at a second timing after the subject ingests the meal;
estimate the second blood glucose level by using the first blood glucose level, the meal information, and the blood glucose level estimation rule; and
generate, by using the reference range and the second blood glucose level, advice information including at least one of advice relating to the meal and advice relating to behavior of the subject after ingesting the meal.

2. The advice generation apparatus according to claim 1, wherein
a plurality of the second timings are set, and
the at least one processor is configured to further execute the instructions to estimate the second blood glucose level at each of the plurality of second timings.

3. The advice generation apparatus according to claim 1, wherein
a plurality of the first timings are set, and
the at least one processor is configured to further execute the instructions to acquire the first blood glucose level at each of the plurality of first timings.

4. The advice generation apparatus according to claim 1, wherein
the at least one processor is configured to further execute the instructions to generate the advice information including advice relating to the meal when the second blood glucose level deviates from the reference range.

5. The advice generation apparatus according to claim 4, wherein
the advice information includes information indicating not ingesting a first item included in the meal.

6. The advice generation apparatus according to claim 4, wherein
the advice information includes an ingestion amount of a second item included in the meal.

7. The advice generation apparatus according to claim 1, wherein
the meal includes a plurality of items, and
the advice information includes ingestion order of the plurality of items.

8. The advice generation apparatus according to claim 7, wherein
the advice information further includes an ingestion interval between two of the items adjacent to each other in ingestion order.

9. The advice generation apparatus according to claim 1, wherein
the first blood glucose level is a blood glucose level before breakfast,
the meal information includes an item included in the meal and also includes information capable of determining calories to be ingested in the meal, and
the blood glucose level estimation rule is indicated by a following equation.
$$The second blood glucose level = the first blood glucose level + ( calories to be ingested in the meal ) \times α$$
where α is a coefficient computed by using the first blood glucose level and the meal information.

10. An advice generation method comprising
performing, by a computer:
acquiring a first blood glucose level being a blood glucose level of a subject at a first timing, meal information being information relating to a meal to be ingested by the subject later than the first timing, and a reference range of a blood glucose level of the subject;
acquiring a blood glucose level estimation rule being set for each of the subjects and estimating, by using the first blood glucose level and the meal information, a second blood glucose level being a blood glucose level at a second timing after the subject ingests the meal;
estimating the second blood glucose level by using the first blood glucose level, the meal information, and the blood glucose level estimation rule; and
generating, by using the reference range and the second blood glucose level, advice information including at least one of advice relating to the meal and advice relating to behavior of the subject after ingesting the meal.

11. A non-transitory computer-readable storage medium storing a program causing a computer to execute:
acquiring a first blood glucose level being a blood glucose level of a subject at a first timing, meal information being information relating to a meal to be ingested by the subject later than the first timing, and a reference range of a blood glucose level of the subject;
acquiring a blood glucose level estimation rule being set for each of the subjects and estimating, by using the first blood glucose level and the meal information, a second blood glucose level being a blood glucose level at a second timing after the subject ingests the meal;
estimating the second blood glucose level by using the first blood glucose level, the meal information, and the blood glucose level estimation rule; and
generating, by using the reference range and the second blood glucose level, advice information including at least one of advice relating to the meal and advice relating to behavior of the subject after ingesting the meal.

12. The advice generation apparatus according to claim 1, wherein
the blood glucose level estimation rule is a model generated by using machine learning, and
the advice information includes advice relating to behavior of the subject after ingesting the meal, thereby allowing the subject to make a decision to keep his or her own blood glucose level within a preferred range.
